(19) United States
(12) Patent Application Publication
(10) Pub. No.: US 2020/0230099 A1
(43) Pub. Date: Jul. 23, 2020

(54) Stable Pharmaceutical Composition Comprising Zanamivir

(71) Applicant: Dae Hwa Pharma. Co., Ltd., Hoengseong-gun Gangwon-do (KR)

(72) Inventors: In Hyun Lee, Buk-gu Gwangju (KR); Min Hee Son, Seongnam-si Gyeonggi-do (KR); Sol Kim, Seongnam-si Gyeonggi-do (KR); Hye Jin Park, Seongnam-si Gyeonggi-do (KR)

(21) Appl. No.: 16/489,957

(22) PCT Filed: Jan. 31, 2018
(86) PCT No.: PCT/KR2018/001327

§ 371 Date: Aug. 29, 2019

(30) Foreign Application Priority Data
Mar. 2, 2017 (KR) 10-2017-0027224
Dec. 20, 2017 (KR) 10-2017-0175681

(51) Int. Cl. A61K 31/351; A61K 31/215; A61K 47/14; A61K 47/10; A61K 47/36

(52) U.S. Cl. CPC A61K 31/351; A61K 31/215; A61K 9/107; A61K 47/10; A61K 47/36; A61K 47/14

(57) ABSTRACT

The present invention relates to an oral pharmaceutical composition comprising zanamivir. Specifically, a pharmaceutical formulation comprising zanamivir as an active ingredient, and triglyceride, an acylglycerol complex and additional non-ionic surfactant, having good absorption rate in body, formulation stability, and content uniformity is provided.

### TECHNICAL FIELD

The present invention relates to an oral pharmaceutical composition comprising an antiviral agent. Specifically, the present invention provides a pharmaceutical formulation with improved absorption rate in the body, stability of formulation, and content uniformity, comprising zanamivir as an active ingredient, and further comprising triglyceride, an acylglycerol compound, and a surfactant.

### BACKGROUND ART

The first generation antivirals such as amantadine or rimantadine are believed to have mechanism blocking M2-protein ion-channel of type A influenza virus. Blocking the influx of H+ ion through the M2-protein channel inhibits viral uncoating and release of free ribonucleoproteins into the cytoplasm. This only occurs in type A strain and not in type B strain. The next antiviral strategy resulted in the development of the second generation antivirals such as zanamivir or oseltamivir, which inhibit either hemagglutinin (HA) or neuraminidase (NA) enzyme forming a mushroom-shaped projection on the surface of influenza A and B viruses. These proteins bind to the membrane surface of a target cell to be infected by cleaving sialic acid residues of sialoglycoprotein and sialoglycolipid. Further, at the end of viral replication, the neuraminidase enzyme is essential for cleaving sialic acid from receptors to allow virus release. Zanamivir is the first neuraminidase inhibitor as commercially available and used for the treatment and prevention of both influenza A and B viruses.

Oseltamivir has chemical structure of formula I below and is commercially available under the trade name of Tamiflu®. It is widely used as a medicine for swine flu and avian influenza.

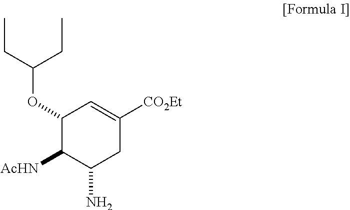

The chemical name of zanamivir is 5-(acetylamino)-4-[(aminoiminomethyl)amino]-2,6-anhydro-3,4,5-trideoxy-D-glycero-D-galacto-non-2-enonic acid (Formula II), and is represented by the following structure:

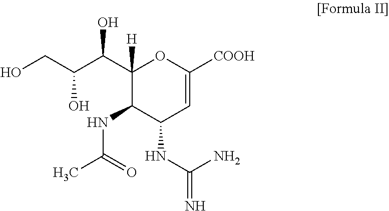 [SMILES: CC(=O)N[C@H]1[C@H]([C@H](O)[C@H](O)CO)OC(C(=O)O)=C[C@@H]1NC(=N)N]

Zanamivir binds to a conserved region of neuraminidase enzyme of influenza virus, which catalyses cleavage of sialic acid residues attached to glycolipids or glycoproteins. It is commercially available under the trade name of RELENZA®.

It is known that bioavailability of zanamivir is generally about 2%, and 4% to 17% when administered through inhalation as powder. 5 mg of inhalant powder is twice inhaled, twice a day for 5 days. RELENZA is currently used in treatment of type A and B influenza, while the use of Diskhaler is required for oral inhalation. As such, the medicinal product is disadvantageous in that additional respirator for oral inhalation is required, it is hard to be administered in a fixed amount every time, and explanation to patients on how to use the appliance is needed. Especially, pediatric use is difficult.

Based on the above, the present inventors intended to improve low bioavailability of zanamivir upon oral administration and to solve problems of inhaled administration, so that an oral formulation with easy administration and improved pharmaceutical properties is finally developed.

### DETAILED DISCLOSURE OF INVENTION

### Technical Problem

Despite excellent antiviral effect, zanamivir was difficult to be widely used because of its low bioavailability when orally administered. When trying different solutions to solve the problem, it was found that zanamivir can be administered with additives to improve absorption rate of zanamivir, based on which the present invention was completed.

The present invention is to provide an oral pharmaceutical composition and a formulation with improved bioavailability upon oral administration and good pharmaceutical properties, and a preparation method thereof.

### Means to be Solved

The present inventors found that when administering zanamivir having low bioavailability upon oral administration along with other additives, the bioavailability for oral administration can be increased.

Specifically, the present inventors used triglyceride, an acylglycerol complex or another non-ionic surfactants as additives to increase absorption rate of zanamivir. With just one or two types of the additives, few improvements on absorption rate were found. However, when all three types of the additives were used, surprisingly, it was found that absorption rate is very high.

Accordingly, the present invention provides a pharmaceutical composition comprising zanamivir as an active ingredient and triglyceride, an acylglycerol complex, and another non-ionic surfactant as additives.

Triglyceride may be one or more selected from the group consisting of triacetin, tripropionin, tributyrin, trivalerin, tricaproin, tricaprylin (Captex8000), tricaprin, triheptanoin, trinonanoin, triundecanoin, trilaurin, tridecanoin, trimyristin, tripentadecanoin, tripalmitin, glyceryl triheptadecanoate, and triolein.

The acylglycerol complex may be one or more selected from the group consisting of glyceryl behenate, glyceryl monooleate (Peceol), glyceryl stearate, and glyceryl palmitostearate.

The non-ionic surfactant may be one or more selected from the group consisting of polyoxyethylene-polyoxypropylene copolymer (poloxamer), sorbitan ester (Span), polyoxyethylene sorbitan (Tween), and polyoxyethylene ether (Brij).

The present inventors also discovered that when triglyceride, an acylglycerol complex, and another non-ionic surfactant are all comprised, phase separation does not occur immediately after preparation or after centrifugation and also the content is uniform.

Especially, the present inventors identified when a pharmaceutical composition comprising zanamivir and triglyceride, an acylglycerol complex, and another non-ionic surfactant as additives is prepared into the form of an emulsion or a syrup, administration is convenient and bioavailability, stability, and content uniformity are all improved.

In addition, the present invention provides an emulsion or a syrup comprising triglyceride, acylglycerol complex and another non-ionic surfactant as additives, upon preparation of a combination comprising zanamivir and oseltamivir.

### Advantageous Effects

The present invention solves a problem that since bioavailability is low when zanamivir is orally administered, inhalation is the only viable option. As such, drug compliance is increased through oral administration and zanamivir can be applied to patients having problems with inhalation. Further, cost can be reduced as respirator is not required, and a variation problem in administered amount upon inhalation has also been solved.

The present invention also provides a pharmaceutical composition or formulation that has not only high bioavailability upon oral administration, but also good pharmaceutical properties such as stability and content uniformity, so that zanamivir can orally be administered.

Especially, the present invention provides an emulsion or a syrup formulation comprising zanamivir, which results in that administration is convenient and bioavailability, stability, and content uniformity are all improved.

The present invention also provides an emulsion or a syrup formulation with good stability, comprising zanamivir and oseltamivir.

### BRIEF DESCRIPTION OF DRAWINGS

FIG. 1 shows the absorption rate of the compositions of Examples 1 to 7.

FIG. 2 depicts the results of phase separation test observed in the compositions of Example 1 to 13 immediately after preparation and after centrifugation.

FIG. 3 shows bioavailability data obtained from intravenous administration of zanamivir dissolved in physiological saline and oral administration of composition from Example 1.

FIG. 4 shows a property of a pharmaceutical composition in a syrup form comprising zanamivir, or zanamivir and oseltamivir.

FIG. 5 shows a concentration change of zanamivir in vivo depending on the dosage.

### BEST MODE CARRYING OUT FOR INVENTION

The present invention relates to a pharmaceutical composition comprising an antiviral agent, triglyceride, an acylglycerol complex, and a non-ionic surfactant.

The antiviral agent of the present invention may be, but is not limited to, one or more selected from the group consisting of zanamivir, tenofovir disoproxil, cidofovir, ganciclovir, foscarnet, ribavirin, oseltamivir, and pharmaceutically acceptable salts thereof.

Especially, the present invention relates to a pharmaceutical composition comprising zanamivir, triglyceride, an acylglycerol complex, and additional non-ionic surfactant.

Triglyceride may be, but is not limited to, one or more selected from the group consisting of triacetin, tripropionin, tributyrin, trivalerin, tricaproin, tricaprylin (Captex8000), tricaprin, triheptanoin, trinonanoin, triundecanoin, trilaurin, tridecanoin, trimyristin, tripentadecanoin, tripalmitin, glyceryl triheptadecanoate, and triolein. Most preferably, triglyceride is tricaprylin (Captex8000).

Triglyceride is comprised in an amount of 1-20 wt %, and more preferably 3-15 wt % based on the weight of zanamivir.

The acylglycerol complex may be, but is not limited to, one or more selected from the group consisting of glyceryl behenate, glyceryl monooleate (Peceol), glyceryl stearate, and glyceryl palmitostearate. Most preferably, the acylglycerol complex is glyceryl monooleate (Peceol).

The acylglycerol complex is comprised in an amount of 1-30 wt %, and more preferably, 5-25 wt % based on the weight of zanamivir

The non-ionic surfactant may be, but is not limited to, one or more selected from the group consisting of polyoxyethylene-polyoxypropylene copolymer (poloxamer), sorbitan ester (Span), polyoxyethylene sorbitan (Tween), and polyoxyethylene ether (Brij). Most preferably, the non-ionic surfactant is polyoxyethylene sorbitan (Tween).

The non-ionic surfactant is comprised in an amount of 1-30 wt %, and more pereferably, 2-25 wt % based on the weight of zanamivir

The composition according to the present invention exerts high bioavailability when triglyceride, an acylglycerol complex, and additional non-ionic surfactant are all comprised.

Specifically, the composition comprising zanamivir as an active ingredient, and Captex8000, Peceol, and Tween80 as additives increases bioavailability of zanamivir and exerts pharmaceutical properties such as good stability and content uniformity.

The composition of the present invention may further comprise a gum. The gum may be, but is not limited to, one or more selected from the group consisting of arabic gum, agar, guar gum, rhamsan gum, tragacanth gum, and xanthan gum. Arabic gum is most preferable.

The composition according to the present invention may further comprise a sugar. The sugar may be one or more selected from the group consisting of sucrose, maltose, lactose, isomaltose, fructooligosaccharide, galactooligosaccharide, isomaltooligosaccharide, maltodextrin, and mannan oligosaccharide.

The present invention comprises a pharmaceutical formulation formulated from the composition above. The formulation may be formulated for oral administration.

The composition of the present invention may be formulated into a dosage form for oral administration such as powders, granules, tablets, capsules, suspensions, emulsions, syrups, and aerosols according to conventional methods. Preferably, it may be an emulsion or a syrup.

The present invention relates to an emulsion or a syrup comprising zanamivir as an active ingredient and triglyceride, an acylglycerol complex, and additional non-ionic surfactant as additives.

Specifically, the present invention relates to an emulsion or a syrup formulation comprising zanamivir as an active ingredient and Captex8000, Peceol, and Tween80 as additives.

The present invention relates to an emulsion or a syrup formulation comprising zanamivir and oseltamivir as active ingredients and triglyceride, an acylglycerol complex, and additional non-ionic surfactant as additives.

Specifically, the present invention relates to an emulsion or a syrup comprising zanamivir and oseltamivir as active ingredients and Captex8000, Peceol, and Tween80 as additives.

The present invention comprises a preparation method comprising the steps of:

a) mixing triglyceride, acylglycerol complex and additional non-ionic surfactant to obtain a mixture;

b) adding the mixture to distilled water in which one or more active ingredients selected from the group consisting of zanamivir and oseltamivir are dissolved, to obtain a solution; and

c) stirring and emulsifying the solution obtained from step b).

The present invention comprises a preparation method comprising the steps of:

a) dissolving sucrose in distilled water to obtain a sucrose solution;

b) adding one or more active ingredients selected from the group consisting of zanamivir and oseltamivir to the sucrose solution, and then stirring; and

c) mixing triglyceride, an acylglycerol complex, and an additional non-ionic surfactant, and then stirring.

Hereinafter, the present invention will be described in more detail through the working examples. However, the examples are provided merely for helping the understanding of the present invention, and the scope of the present invention is not limited to the following examples.

### Example 1

According to the ingredients and amounts in Table 1 below, an oral pharmaceutical composition comprising zanamivir was prepared.

Captex™ 8000 (Abitec), PECEOL™ (Gattefosse), and Tween™ 80 (NOF) were mixed according to the amount in Table 1, followed by adding into a distilled water where zanmivir was completely dissolved. Then, oil phase and water phase were mixed in a certain ratio and stirring at 25□ for 2 hours to obtain an emulsion.

### Example 2

In the same manner as Example 1, an oral pharmaceutical composition comprising zanamivir was prepared using Captex™ 8000 alone.

### Example 3

In the same manner as Example 1, an oral pharmaceutical composition comprising zanamivir was prepared using PECEOL™ alone.

### Example 4

In the same manner as Example 1, an oral pharmaceutical composition comprising zanamivir was prepared using Tween™ 80 alone.

### Example 5

In the same manner as Example 1, an oral pharmaceutical composition was prepared using Captex™ 8000 and PECEOL™ wherein they are mixed in a ratio shown in Table 1, and then adding to a distilled water where zanmivir is completely dissolved.

### Example 6

In the same manner as Example 1, an oral pharmaceutical composition was prepared using PECEOL™ and Tween™ 80 wherein they are mixed in a ratio shown in Table 1, and then adding to a distilled water where zanmivir is completely dissolved.

### Example 7

In the same manner as Example 1, an oral pharmaceutical composition comprising zanamivir was prepared using Captex™ 8000 and Tween™ 80 wherein they are mixed in a ratio shown in Table 1, and then adding to a distilled water where zanmivir is completely dissolved.

TABLE 1

| | Ingredient ratio (v/v, %) | | |
|---|---|---|---|
| Example | Captex ™ 8000 | PECEOL ™ | Tween ™ 80 |
| 1 | 27.78 | 55.56 | 16.67 |
| 2 | 100 | — | — |
| 3 | — | 100 | — |
| 4 | — | — | 100 |
| 5 | 50 | 50 | — |
| 6 | — | 50 | 50 |
| 7 | 50 | — | 50 |

### Examples 8 to 13

According to the ingredients and amounts in Table 2 below, an oral pharmaceutical composition comprising an antiviral agent was prepared.

Captex™ 8000 (Abitec), PECEOL™ (Gattefosse), and Tween™ 80 (NOF) were mixed in the ratio shown in Table 2, followed by adding into a distilled water where an antiviral agent was completely dissolved. Then, oil phase and water phase were mixed in a certain ratio and stirring at 25□ for 2 hours to obtain an emulsion. The concentration of the antiviral agent was 10 mg/mL in all formulations of Examples 8 to 13.

TABLE 2

| | | Ingredient ratio (v/v, %) | | |
|---|---|---|---|---|
| | | Captex ™ | | Tween ™ |
| Example | Antiviral Agent | 8000 | PECEOL ™ | 80 |
| 8 | Tenofovir disoproxil | 27.78 | 55.56 | 16.67 |
| 9 | Cidofovir | | | |
| 10 | Ganciclovir | | | |
| 11 | Foscarnet | | | |
| 12 | Ribavirin | | | |
| 13 | Oseltamivir | | | |

### Example 14

### Preparation of a Syrup Formulation

A syrup formulation comprising zanamivir was prepared according the ingredients and amounts in Table 3 below

A certain amount of sucrose was weighed and dissolved in distilled water. A certain amount of zanamivir was added into the sucrose solution, and then stirred for 2 hours at 25□ and 1,000 rpm.

Once it was completely and clearly dissolved, Captex™ 8000 (Abitec), PECEOL™ (Gattefosse), and Tween™ 80 (NOF) were mixed according to the ratio in Table 3, and then stirred at 1,000 rpm to obtain a syrup formulation.

TABLE 3

| Concentra- | Excipients (W/W %) | | | | |
|---|---|---|---|---|---|
| tion of | | Distilled | Captex ™ | | Tween ™ |
| zanamivir | Sucrose | Water | 8000 | PECEOL ™ | 80 |
| 5 mg/ml | 40 | 20 | 10.4 | 20.8 | 6.3 |

### Example 15

### Preparation of a Combination of Zanamivir and Oseltamivir

A syrup formulation comprising zanamivir and oseltamivir was prepared according to the ingredients and amounts in Table 4 below.

A certain amount of sucrose was weighed and dissolved in distilled water. Zanamivir and oseltamivir were added into the sucrose solution, and then stirred for 2 hours at 25□ and 1,000 rpm.

Once it was completely and transparently dissolved, Captex™ 8000 (Abitec), PECEOL™ (Gattefosse), and Tween™ 80 (NOF) were mixed according to the ratio in Table 4, and then stirred at 1,000 rpm to obtain a syrup formulation.

TABLE 4

| | | Excipients (W/W, %) | | | | |
|---|---|---|---|---|---|---|
| Concentration of | Concentration of | | Distilled | Captex ™ | | Tween ™ |
| zanamivir | oseltamivir | Sucrose | Water | 8000 | PECEOL ™ | 80 |
| 5 mg/ml | 10 mg/ml | 40 | 20 | 10.4 | 20.8 | 6.3 |

### Test Example 1

### Absorption Rate Evaluation

An oral pharmaceutical composition comprising zanamivir prepared from each example was orally administered to ICR mice (6-week, female) in dosage of 50 mg/kg and using gastric sonde. At 0 min, 30 min, lhr, 2 hr, 4 hr, 6 hr, and 8 hr after administration of a drug, blood was taken from orbital vein and centrifuged at 8,000×g and 4□ for 20 minutes to obtain a plasma sample, followed by storing at −70□.

After the plasma sample was melted at room temperature, it was stirred using vortex mixer for a minute. 200.0 μL of 70% acetonitrile and 300.0 μL of 60% acetonitrile were put in 100.0 μL of the sample, and stirred using vortex mixer for 5 minutes at 3,000 rpm. Each sample was centrifuged at 14,000×g and 4□ for 20 minutes, and then 300.0 μL of supernatant was taken and filtered using syringe filter (PTFE, chromdisc, 13 mm, pore size 0.20 mm). 200.0 μL of filtrate was taken and analyzed with HPLC.

The pharmacokinetics parameters as calculated based on the blood concentration of zanamivir obtained above are shown in Table 5 below.

TABLE 5

| Example | Cₘₐₓ(μg/ml) | Tₘₐₓ(hr) | AUC_{0-T}(min · μg/ml) |
|---|---|---|---|
| 1 | 1.69 ± 0.68 | 2 | 508.15 |
| 2 | 0.34 ± 0.06 | 1 | 118.32 |
| 3 | 0.15 ± 0.12 | 0.5 | 30.81 |
| 4 | 0.29 ± 0.15 | 6 | 127.01 |
| 5 | 0.38 ± 0.1 | 8 | 148.01 |
| 6 | 0.29 ± 0.01 | 0.5 | 120.67 |
| 7 | 0.23 ± 0.04 | 0.5 | 94.61 |

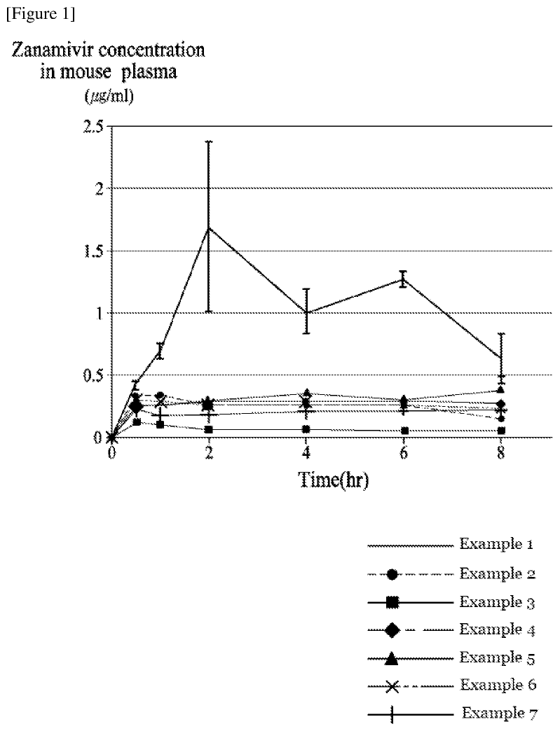

The result of blood concentration test is presented in Table 6 and FIG. 1. As shown in Table 6 below and FIG. 1, it was confirmed that Example 1 exerted the highest absorption rate. This means that the absorption rate of the composition comprising all of the three additives is most remarkable.

TABLE 6

| Time | Exam- | Exam- | Exam- | Exam- | Exam- | Exam- | Exam- |
|---|---|---|---|---|---|---|---|
| (hr) | ple 1 | ple 2 | ple 3 | ple 4 | ple 5 | ple 6 | ple 7 |
| 0 | 0 | 0 | 0 | 0 | 0 | 0 | 0 |
| 2 | 1.69 | 0.26 | 0.06 | 0.27 | 0.29 | 0.26 | 0.18 |
| 4 | 1.01 | 0.28 | 0.07 | 0.26 | 0.36 | 0.26 | 0.21 |
| 6 | 1.27 | 0.23 | 0.05 | 0.29 | 0.29 | 0.26 | 0.21 |
| 8 | 0.63 | 0.15 | 0.05 | 0.27 | 0.38 | 0.23 | 0.22 |

### Test Example 2

### Absorption Rate Evaluation of a Syrup Ormulation

Using a syrup formulation comprising zanamivir prepared in Example 14, the absorption rate was evaluated in the same manner as Test Example 1.

The dosage of zanamivir was 50 mg/kg or 100 mg/kg. Blood was taken and measured at 0 min, 30 min, 1 hr, 2 hr, and 4 hr.

The pharmacokinetics parameters as calculated based on the blood concentration of zanamivir obtained above are shown in Table 7 below.

TABLE 7

| | Cₘₐₓ | Tₘₐₓ | AUC_{0-4 hr} |
|---|---|---|---|
| Dosage | (μg/ml) | (hr) | (min · μg/ml) |
| 50 mg/kg | 0.36 ± 0.013 | 0.5 | 55.8 |
| 100 mg/kg | 0.71 ± 0.15 | 0.5 | 78 |

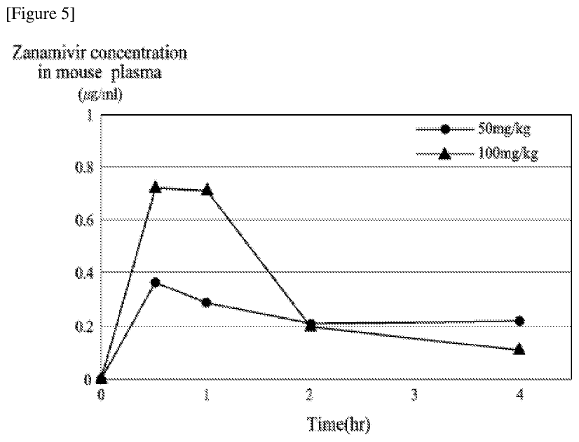

As shown in Table 7 above and FIG. 5, Cₘₐₓ and AUC₀₋₄ₕᵣ increased as the oral dosage increased.

### Test Example 3

### Stability Test

The formulations were prepared according to Examples 1 to 7 and examined for their properties, followed by centrifuging at 1,500×g and 20□ for 15 minutes. Immediately after centrifugation, it was examined whether phase separation was observed to evaluate the stability of the formulation.

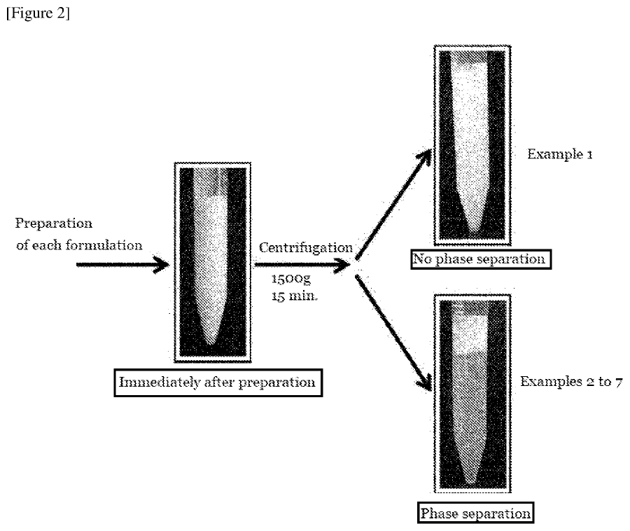

As shown in FIG. 2, there was no phase separation immediately after the preparation in Examples 1 to 7. No phase separation was observed in Example 1 after centrifugation, while phase separation occurred in Examples 2 to 7.

As such, it was confirmed that only Example 1 has stability in showing no phase separation immediately after preparation and after centrifugation.

### Test Example 4

### Content Uniformity Test

After centrifugation according to Test Example 3, the formulation of Example 1 was analyzed on the content of zanamivir in the top, middle, and bottom layers. The results are shown in Table 8.

TABLE 8

| Initial content of | Content after centrifugation (%) | | |
|---|---|---|---|
| Zanamivir (%) | Top | Middle | Bottom |
| 100 | 101.788 | 97.455 | 102.655 |

As shown in Table 8, it was confirmed that the content of zanamivir existed uniformly over the top, middle, and bottom layers even after centrifugation of Example 1.

### Test Example 5

### Stability Test of a Syrup Formulation

A syrup formulation comprising zanamivir prepared in Example 14 was stored for 0 and 40 days at 25□, and the property and content were analyzed. The results are shown in Table 9 below.

TABLE 9

| | |
|---|---|
| Storage Time | Zanamivir content (%) |

| | | |
|---|---|---|
| 0 | day | 97.4 ± 0.18 |
| 40 | days | 96.6 ± 0.57 |

Upon evaluation of the content test of the syrup formulation, as shown in Table 9 above, it was confirmed that the formulation stored for more than 40 days maintained the content compared to the formulation at initial stage, which resulted in that the formulation is stable.

Moreover, phenomenon such as phase separation or precipitation was not observed from the syrup formulation at initial stage as well as after stored. As such, it was confirmed that the formulation is stable.

### Test Example 6

### Additives Ratio Test

Evaluation on stability and content uniformity from Test Example 3 and 4 was repeatedly performed on the different ratios of the additives. Table 10 shows the ratio of the additives exerting good stability and content uniformity.

TABLE 10
Weight (%) based on Zanamivir

| Captex ™ 8000 | PECEOL ™ | Tween ™ 80 |
|---|---|---|
| 1-20 | 1-30 | 1-30 |

It was confirmed that stability and content uniformity are good when 1-20 wt % of Captex™ 8000, 1-30 wt % of PECEOL™, and 1-30 wt % of Tween™ 80 are comprised based on the weight of zanmivir.

### Test Example 7

### Bioavailability Test

The test result of Test Example 1 obtained from oral administration of the composition of Example 1 to mice, and the results obtained from oral administration and intravenous administration of a solution to mice where 10 mg/ml of zanamivir was dissolved in physiological saline are shown in Table 11.

TABLE 11

| | Administration | AUC_{0-T} | C max | Tmax | Bioavailability |
|---|---|---|---|---|---|
| | rout | (min · μg/ml)ₑ | (μg/ml) | (hr) | (%) |
| Composition of | Oral | 508.15 | 1.69 ± 0.68 | 2 | 43.13 |
| Example 1 | | | | | |
| Composition of | Oral | — | — | — | — |
| zanamivir | Intravenous | 1178.11 | 76.35 ± 12.45 | 0.083 | — |
| dissolved in | | | | | |
| physiological | | | | | |
| saline | | | | | |

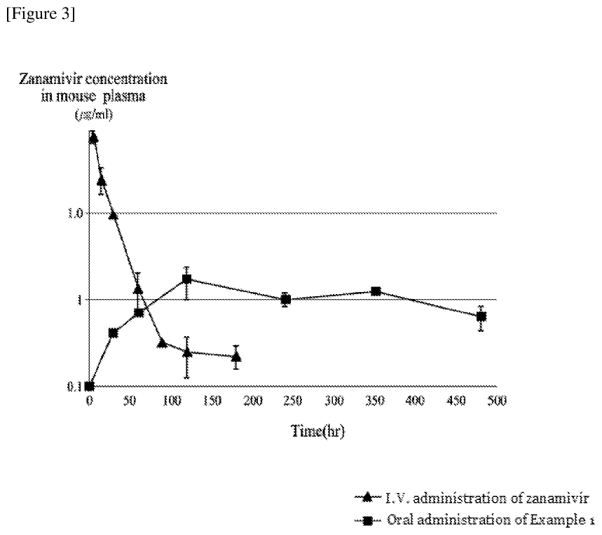

No absorption in body was found in the oral administration of zanamivir dissolved in physiological saline. Further, as shown in FIG. 3, the bioavailability decreased rapidly in the intravenous administration of zanamivir dissolved in physiological saline, while the oral administration of the composition of Example 1 exerts good bioavailability.

### Test Example 8

### Stability Test

The formulations were prepared according to Examples 8 to 13 and examined for their properties, followed by centrifuging at 1,500×g and 20□ for 15 minutes. Immediately after centrifugation, it was examined whether phase separation was observed to evaluate the stability of the formulation.

As shown in FIG. 2, there was no phase separation immediately after the preparation in the formulations of Examples 8 to 13. Even after centrifugation, no phase separation occurred in the formulations of Examples 8 to 13, from which the stability of the formulations was confirmed.

### Test Example 9

### Stability Test of a Syrup Formulation

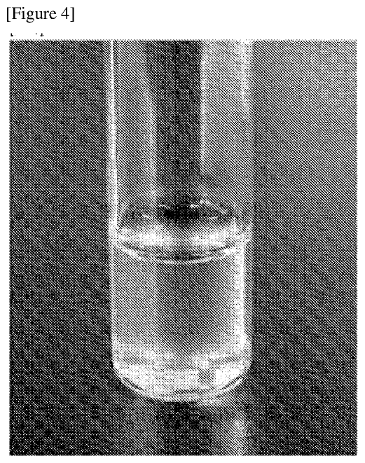

As shown in FIG. 4, it was confirmed that a syrup of zanamivir and a combination syrup of zanamivir and oseltamivir, which were prepared in Examples 14 and 15, respectively, have clear and transparent properties.

### Test Example 10

### Content Uniformity Test

After centrifugation in Test Example 6, the formulations from Example 8 to 13 were analyzed on the contents in the top, middle, and bottom layers. The results are shown in Table 12.

TABLE 12

| | Content after Centrifugation | | |
|---|---|---|---|
| | (%, initial substrate content 100%) | | |
| Example | Top | Middle | Bottom |
| 8 | 99.89 | 100.06 | 101.02 |
| 9 | 100.25 | 100.98 | 99.78 |
| 10 | 101.56 | 100.12 | 99.87 |
| 11 | 98.45 | 101.54 | 100.12 |
| 12 | 100.21 | 100.57 | 99.61 |
| 13 | 101.54 | 99.45 | 99.63 |

As shown in Table 12, it was confirmed that the content of zanamivir existed uniformly over the top, middle, and bottom layers even after centrifugation of the formulations of Examples 8 to 13.

1. A pharmaceutical composition comprising an antiviral agent, triglyceride, an acylglycerol complex, and a non-ionic surfactant.

2. The pharmaceutical composition according to claim 1, characterized in that the antiviral agent is one or more selected from the group consisting of zanamivir, tenofovir disoproxil, cidofovir, ganciclovir, foscarnet, ribavirin, oseltamivir, and pharmaceutically acceptable salts thereof.

3. The pharmaceutical composition according to claim 2, characterized in that the antiviral agent is zanamivir or oseltamivir.

4. The pharmaceutical composition according to claim 1, characterized in that the triglyceride is one or more selected from the group consisting of triacetin, tripropionin, tributyrin, trivalerin, tricaproin, tricaprylin, tricaprin, triheptanoin, trinonanoin, triundecanoin, trilaurin, tridecanoin, trimyristin, tripentadecanoin, tripalmitin, glyceryl triheptadecanoate, and triolein.

5. The pharmaceutical composition according to claim 1, characterized in that the acylglycerol complex is one or more selected from the group consisting of glyceryl behenate, glyceryl monooleate, glyceryl stearate, and glyceryl palmitostearate.

6. The pharmaceutical composition according to claim 1, characterized in that the non-ionic surfactant is one or more selected from the group consisting of polyoxyethylene-polyoxypropylene copolymer, sorbitan ester, polyoxyethylene sorbitan, and polyoxyethylene ether.

7. The pharmaceutical composition according to claim 4, characterized in that the triglyceride is tricaprylin.

8. The pharmaceutical composition according to claim 5, characterized in that the acylglycerol complex is glyceryl monooleate.

9. The pharmaceutical composition according to claim 6, characterized in that the non-ionic surfactant is polyoxyethylene sorbitan.

10. The pharmaceutical composition according to claim 3, characterized in that the triglyceride is comprised in an amount of 1-20 wt % based on the weight of zanamivir.

11. The pharmaceutical composition according to claim 3, characterized in that the acylglycerol complex is comprised in an amount of 1-30 wt % based on the weight of zanamivir.

12. The pharmaceutical composition according to claim 3, characterized in that the non-ionic surfactant is comprised in an amount of 1-30 wt % based on the weight of zanamivir.

13. The pharmaceutical composition according to claim 1, comprising zanamivir, tricaprylin, glyceryl monooleate, and polyoxyethylene sorbitan.

14. The pharmaceutical composition according to claim 13, comprising 1-20 wt % of tricaprylin, 1-30 wt % of glyceryl monooleate, and 1-30 wt % of polyoxyethylene sorbitan based on the weight of zanamivir.

15. The pharmaceutical composition according to claim 1, comprising zanamivir, oseltamivir, tricaprylin, glyceryl monooleate, and polyoxyethylene sorbitan.

16. The pharmaceutical composition according to claim 1, further comprising gums or sugars.

17. An oral pharmaceutical formulation, wherein the formulation is formulated from the composition according to claim 1.

18. The pharmaceutical formulation according to claim 17, characterized in that the pharmaceutical formulation is in the form of an emulsion or a syrup.

19. A method for preparing an oral pharmaceutical composition, comprising the steps of:
a) mixing triglyceride, an acylglycerol complex, and a non-ionic surfactant to obtain a mixture;
b) adding the mixture to distilled water in which one or more active ingredients selected from the group consisting of zanamavir and oseltamivir are dissolved to obtain a solution; and
c) stirring and emulsifying the solution obtained from step b).

20. A method for preparing a syrup formulation, comprising the steps of:
a) dissolving sucrose in distilled water to obtain a sucrose solution;
b) adding one or more active ingredients selected from the group consisting of zanamavir and oseltamivir to the sucrose solution, and then stirring; and
c) mixing triglyceride, an acylglycerol complex, and an additional non-ionic surfactant, and then stirring.
